SEQUENCE LISTING (45 sequences)

SEQ ID NO: 1 - DNA, 30 bp, artificial sequence
  HPV-6 specific oligonucleotide probe
aactacatcttccacatacaccaactgcac

SEQ ID NO: 2 - DNA, 30 bp, artificial sequence
  HPV-11 specific oligonucleotide probe
gtctaaatctgctacatacactaaatccgt

SEQ ID NO: 3 - DNA, 30 bp, artificial sequence
  HPV-16(a) specific oligonucleotide probe
atgtgctgccatatctacttcagaatctgt

SEQ ID NO: 4 - DNA, 30 bp, artificial sequence
  HPV-18 specific oligonucleotide probe
tacacagtctcctgtacctgggcagtcatt

SEQ ID NO: 5 - DNA, 30 bp, artificial sequence
  HPV-26 specific oligonucleotide probe
ttatctgcagcatctgcatccacttgcttc

SEQ ID NO: 6 - DNA, 30 bp, artificial sequence
  HPV-31 specific oligonucleotide probe
tgctgcaattgcaaacagtgatacagtaca

SEQ ID NO: 7 - DNA, 30 bp, artificial sequence
  HPV-32 specific oligonucleotide probe
caactgaagacacatacaagtctatgtttg

SEQ ID NO: 8 - DNA, 30 bp, artificial sequence
  HPV-33 specific oligonucleotide probe
cacacaagtaactagtgacagtacctgtaa

SEQ ID NO: 9 - DNA, 30 bp, artificial sequence
  HPV-35 specific oligonucleotide probe
gtgttctgctgtgtcttctagtgatttatg

SEQ ID NO: 10 - DNA, 30 bp, artificial sequence
  HPV-39 specific oligonucleotide probe
tctatagagtcttccataccttctgtctgt

SEQ ID NO: 11 - DNA, 30 bp, artificial sequence
  HPV-42 specific oligonucleotide probe
catctggtgatacatatacagctgtctacc

SEQ ID NO: 12 - DNA, 30 bp, artificial sequence
  HPV-43 specific oligonucleotide probe
gaccctactgtgcccagtacatatctgcaa

SEQ ID NO: 13 - DNA, 30 bp, artificial sequence
  HPV-44 specific oligonucleotide probe
acacagtcccctccgtctacatattctact

SEQ ID NO: 14 - DNA, 30 bp, artificial sequence
  HPV-45 specific oligonucleotide probe
aatcctgtgccaagtacatatgacgccact

SEQ ID NO: 15 - DNA, 30 bp, artificial sequence
  HPV-51 specific oligonucleotide probe
gccactgctgcggtttccccaacaacacaa

SEQ ID NO: 16 - DNA, 30 bp, artificial sequence
  HPV-52 specific oligonucleotide probe
ggttaaaaaggaaagcacatataaagcact

SEQ ID NO: 17 - DNA, 30 bp, artificial sequence
  HPV-53 specific oligonucleotide probe
gtctacatataattcaaagcaaattgctga

SEQ ID NO: 18 - DNA, 30 bp, artificial sequence
  HPV-54 specific oligonucleotide probe
atccacgcaggatagctttaataagtctat

SEQ ID NO: 19 - DNA, 30 bp, artificial sequence
  HPV-55 specific oligonucleotide probe
caactcagtctccatctacaacattacagc

SEQ ID NO: 20 - DNA, 30 bp, artificial sequence
  HPV-56 specific oligonucleotide probe
ctacagaacagttaagtaaatatgctgcta

SEQ ID NO: 21 - DNA, 30 bp, artificial sequence
  HPV-58 specific oligonucleotide probe
cactgaagtaactaaggaaggtacgtactg

SEQ ID NO: 22 - DNA, 30 bp, artificial sequence
  HPV-59 specific oligonucleotide probe
acttcttctattcctaatgtatacattatg

SEQ ID NO: 23 - DNA, 30 bp, artificial sequence
  HPV-61 specific oligonucleotide probe
tacatccccccctgtatctgaatatctact

SEQ ID NO: 24 - DNA, 22 bp, artificial sequence
  HPV-62 specific oligonucleotide probe
cgcctccactgctgcatactgc

SEQ ID NO: 25 - DNA, 30 bp, artificial sequence
  HPV-66 specific oligonucleotide probe
tgcagctaaaagcacattaactaattgtac

SEQ ID NO: 26 - DNA, 30 bp, artificial sequence
  HPV-67 specific oligonucleotide probe
tgaggaaaaatcagaggctacatatattaa

SEQ ID NO: 27 - DNA, 30 bp, artificial sequence
  HPV-68 specific oligonucleotide probe
actactgaatcagctgtaccaaatatgttc

SEQ ID NO: 28 - DNA, 30 bp, artificial sequence
  HPV-69 specific oligonucleotide probe
ctgcatctgccacttttaaaccattctact

SEQ ID NO: 29 - DNA, 30 bp, artificial sequence
  HPV-70 specific oligonucleotide probe
gcaccgaaacggccatacctgctgcacaat

SEQ ID NO: 30 - DNA, 30 bp, artificial sequence
  HPV-72 specific oligonucleotide probe
agcgtcctctgtatcagaatatacctgcct

SEQ ID NO: 31 - DNA, 30 bp, artificial sequence
  HPV-74 specific oligonucleotide probe
tactacacaatcccctcctgctgctgccac

SEQ ID NO: 32 - DNA, 30 bp, artificial sequence
  HPV-MM4(82) specific oligonucleotide probe
ctgttactcaatctgttgcacaaatgcgcc

SEQ ID NO: 33 - DNA, 30 bp, artificial sequence
  HPV-MM7(83) specific oligonucleotide probe
ctgctacacaggctaatgaatacagcactg

SEQ ID NO: 34 - DNA, 30 bp, artificial sequence
  HPV-MM8(84) specific oligonucleotide probe
ctaccaacaccgaatcagaatatacagctg

SEQ ID NO: 35 - DNA, 30 bp, artificial sequence
  HPV-cp8061(71) specific oligonucleotide probe
ccaaaactgttgagtctacatatagtgctg

SEQ ID NO: 36 - DNA, 30 bp, artificial sequence
  HPV-cp8304(81) specific oligonucleotide probe
ctacatctgctgctgcagaatacagtgcta

SEQ ID NO: 37 - DNA, 32 bp, artificial sequence
  HPV-Pan I specific oligonucleotide probe
actgtkgtrgatacyacycgyagtacgcacag

SEQ ID NO: 38 - DNA, 18 bp, artificial sequence
  HPV-Pan II specific oligonucleotide probe
ggcatttgytggtttgtt

SEQ ID NO: 39 - DNA, 25 bp, artificial sequence
  GAPDH nucleic acid probe2 for internal control
caccaactgcttagcacccaayaat

SEQ ID NO: 40 - DNA, 20 bp, artificial sequence
  MY11 F-primer for nucleic acid amplification
cmcagggwcataayaatggg

SEQ ID NO: 41 - DNA, 29 bp, artificial sequence
  biotin-HPV-R1 primer
ataaaytgyaaatcatattcytctgaaaa

SEQ ID NO: 42 - DNA, 20 bp, artificial sequence
  GAPDH-F2 primer
cccatgttcgtcatgggtgt

SEQ ID NO: 43 - DNA, 19 bp, artificial sequence
  biotin-GAPDH-R2 primer
catgagtccttccacgata

SEQ ID NO: 44 - DNA, 1185 bp, Enterobacteria phage T4
catatgagcgacctgaaaagccgtctgattaaggcgagcaccagcaaactgaccgcggaactgaccgcgagcaaattcttcaacgaaaaagatgtggttcgtaccaaaatcccgatgatgaacattgagctgagcggcgagatcaccggtggcatgcagagcggcctgctgattctggcgggtccgagcaagagcttcaagagcaactttggtctgacgatggtgagcagctacatgcgtcaatatccggatgcggtttgcctgttctacgacagcgaatttggcatcaccccggcgtatctgcgtagcatgggtgttgatccggagcgtgtgattcacaccccggttcagagcctggaacaactgcgtatcgatatggtgaaccagctggacgcgattgagcgtggcgaaaaagtggttgtgtttatcgacagcctgggtaacctggcgagcaagaaggaaaccgaagatgcgctgaacgagaaggttgtgagcgacatgacccgtgcgaaaaccatgaagagcctgtttcgtatcgttaccccgtacttcagcaccaaaaacattccgtgcatcgcgattaaccacacctacgaaacccaggaaatgtttagcaagaccgtgatgggtggtggcaccggtccgatgtacagcgcggataccgtttttatcattggtaaacgtcaaatcaaggacggtagcgacctgcaaggttatcaattcgttctgaacgtggagaaaagccgtaccgtgaaggagaagagcaagttctttatcgacgttaagttcgatggcggtattgacccgtacagcggcctgctggacatggcgctggaactgggctttgttgtgaaaccgaagaacggttggtatgcgcgtgagttcctggatgaggaaaccggtgaaatgattcgtgaggagaagagctggcgtgcgaaggacaccaactgcaccaccttctggggcccgctgtttaaacaccagccgttccgtgacgcgatcaagcgtgcgtaccaactgggtgcgatcgatagcaacgagattgttgaggcggaagtggacgaactgattaacagcaaggtggagaaattcaagagcccggaaagcaaaagcaagagcgcggcggacctggaaaccgacctggaacaactgagcgacatggaggaatttaatgaataaggatcc

SEQ ID NO: 45 - protein, 391 aa, Enterobacteria phage T4
MSDLKSRLIKASTSKLTAELTASKFFNEKDVVRTKIPMMNIELSGEITGGMQSGLLILAGPSKSFKSNFGLTMVSSYMRQYPDAVCLFYDSEFGITPAYLRSMGVDPERVIHTPVQSLEQLRIDMVNQLDAIERGEKVVVFIDSLGNLASKKETEDALNEKVVSDMTRAKTMKSLFRIVTPYFSTKNIPCIAINHTYETQEMFSKTVMGGGTGPMYSADTVFIIGKRQIKDGSDLQGYQFVLNVEKSRTVKEKSKFFIDVKFDGGIDPYSGLLDMALELGFVVKPKNGWYAREFLDEETGEMIREEKSWRAKDTNCTTFWGPLFKHQPFRDAIKRAYQLGAIDSNEIVEAEVDELINSKVEKFKSPESKSKSAADLETDLEQLSDMEEFNE

(19) United States
(12) Patent Application Publication
(10) Pub. No.: US 2019/0112646 A1
(43) Pub. Date: Apr. 18, 2019

(54) METHOD OF UTILIZING ENZYME FOR ISOTHERMAL NUCLEIC ACID HYBRIDIZATION

(71) Applicant: GENPRONEX BIOMEDICAL INC., Taipei City (TW)

(72) Inventors: ERR-CHENG CHAN, TAIPEI CITY (TW); YUNG-BIN KUO, TAIPEI CITY (TW)

(21) Appl. No.: 15/869,831

(22) Filed: Jan. 12, 2018

(30) Foreign Application Priority Data
Oct. 12, 2017 (CN) 201710948491.6

(51) Int. Cl. C12Q 1/6832; C12Q 1/689; C12Q 1/70

(52) U.S. Cl. CPC C12Q 1/6832; C12Q 1/708; C12Q 1/689

(57) ABSTRACT

The invention provides a method utilizing an enzyme to proceed isothermal nucleic acid hybridization. The invention uses the biological property of enzyme to replace the conventional heating process for denature or separating double-stranded nucleic acid. By practicing this invention, can a to-be-analyzed double-stranded nucleic acid, a correspondent specific nucleic acid probe and the enzyme be mixed together, and the nucleic acid hybridization can be achieved under constant temperature condition without multiple steps; furthermore, multiple targets hybridization reaction can be performed simultaneously.

This application claims priority for China patent application no. 201710948491.6 filed on Oct. 12, 2017, the content of which is incorporated by reference in its entirety.

### BACKGROUND OF THE INVENTION

### Field of the Invention

The present invention relates to a novel method of nucleic acid hybridization, specifically, the method utilizes an enzyme to proceed isothermal nucleic acid hybridization. The enzyme is recombinase whose biological property enables the method to replace the conventional heating process for denature or separating double-stranded nucleic acid.

### Description of the Prior Art

Nucleic acid hybridization is a technique in which single-stranded nucleic acids are allowed to interact to form complexes, or hybrids with sufficiently similar complementary sequences. This technique allows the detection of specific sequences or may be used to assess the degree of sequence identity. Hybridization may be carried out in solution or more commonly on a solid-phase support, e.g., nitrocellulose paper. Hybridization can be performed with combinations of DNA-DNA (heat-denatured to produce single strands), DNA-RNA, or RNA-RNA molecules.

Common hybridization types on the solid-phase support comprise colony in situ hybridization, dot blotting, Southern blotting, Northern blotting, tissue in situ hybridization and genome in situ hybridization. Conventional solid-phase membrane nucleic acid hybridization comprises following steps: 1. DNA denatured by high temperature (100° C.) for 10 minutes; 2. single strand DNA cooling off instantly; 3. single strand DNA fixed on a nitrocellulose membrane (it takes 5-7 hours); 4. pre-hybridization under 68° C. for 3-12 hours; 5. nucleic acid hybridization under 60-70° C. for 4-20 hours; 6. membrane washing under room temperature for 2-3 hours; 7. color reaction with a marker labeled on a probe.

To conclude, the conventional nucleic acid hybridization method requires temperature control to achieve denaturation and hybridizing, in other words, several steps are needed and hence take lots of time; meanwhile, melting temperature (Tm) of the probe should also be taken into consideration, so it is unlike to perform multiple targets hybridization reaction at the same time. Consequently, it is necessary to develop a novel method to proceed nucleic acid hybridization in which the steps or processes can be simplified, and furthermore the novel method of nucleic acid hybridization has features of time and cost saving as well as performing multiple targets hybridization reaction simultaneously.

### SUMMARY OF THE INVENTION

To improve and simplify the steps of the conventional nucleic acid hybridization method which requires different temperature processes, this present invention provides a method of utilizing enzyme to proceed isothermal nucleic acid hybridization.

The main compositions used in this invention comprise a recombinase and a reaction solution for hybridization. The method of utilizing enzyme for isothermal nucleic acid hybridization essentially comprises the following steps: mix a nucleic acid to be tested, a nucleic acid probe, the recombinase and the reaction solution for hybridization, wherein the nucleic acid probe is complementary to a certain sequence of the nucleic acid to be tested, and the length of the nucleic acid probe is between 15 to 100 nucleotides, preferably between 20 to 60 nucleotides; the recombinase is used for annealing of the nucleic acid probe and the complementary sequence of the nucleic acid to be tested to form a complex of “nucleic acid probe—nucleic acid to be tested” under constant low temperature condition. The recombinase enables DNA-DNA or RNA-DNA hybridization to proceed based on its capability of homologous nucleic acid base pairing and nucleic acid strand exchange.

Recombinase can be isolated or purified from prokaryotes or eukaryotes, and there are two types of recombinase, one is wild type (Shibata T. et al., Method in Enzymology, 100:197 (1983)) and the other is mutant types, such as RecA 441 (Kawashima H. et al., Mol. Gen. Genet, 193:288 (1984)), uvsX protein (Yoncsaki T. et al., Eur. J. Biochem., 148:127 (1985)), Bacillus suhilis RecA protein (Lovett C. M. et al., J. Biol. Chem., 260:3305 (1985)), Ustilago Reel protein (Kmiec E. B. et al., Cell, 29:367 (1982)), Thermus aquaticus or Thermus thermophilus RecA-like protein (Angov E. et al., J. Bacteriol., 176:1405 (1994); Kato R. et al., J. Biochem., 114:926 (1993)) or yeast, mouse, human-derived RecA-like protein (Shinohara A. et al., Nature Genetics, 4:239 (1993)) and others, for instance, Rad51, Rad51B, Rad51C, Rad51D, Rad51E, XRCC2 or DMC1.

The recombinase in the invention can be utilize in liquid solution or solid matrix (plastics, paper, glass, magnetic bead, nylon or nitrocellulose). The nucleic acid probe or the nucleic acid to be tested can be fixed on the surface of a solid matrix for nucleic acid hybridization. Methods for proceeding nucleic acid hybridization comprise in situ hybridization, Southern blotting, Northern blotting and microarray hybridization. The temperature for hybridization is between 30 to 50° C., preferably between 30 to 45° C., and the duration of hybridization is between 5 to 60 minutes, preferably between 10 to 60 minutes.

The distinguishing feature and effect of this invention include proceeding nucleic acid hybridization under constant low temperature environment, lowering the need for related instruments and materials and simultaneously mixing the nucleic acid to be tested and the nucleic acid probe. It takes only 30 to 60 minutes to perform this invention, and more probe hybridization reactions may be carried out at the same time.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings are included to provide a further understanding of the invention, and are incorporated in and constitute a part of this specification.

The drawings illustrate embodiments of the invention and, together with the description, serve to explain the features and advantages of the invention. In the drawings:

FIG. 1 is a flowchart illustrating method of utilizing enzyme for isothermal nucleic acid hybridization in the invention;

FIG. 2 is a schematic drawing of the distribution of nucleic acid to be tested in the embodiment 1;

FIG. 3A to 3D are result pictures of utilizing enzyme for DNA-DNA hybridization in the embodiment 1;

FIG. 4 is a result picture of utilizing enzyme for nucleic acid hybridization to identify HPV type 52 in the embodiment 2;

FIG. 5 is a result picture of utilizing enzyme for nucleic acid hybridization to identify HPV types 33 and 58 in the embodiment 2; and

FIG. 6 is a result picture of utilizing enzyme for nucleic acid hybridization to identify HPV type 56 in the embodiment 2.

### DESCRIPTION OF THE PREFERRED EMBODIMENTS

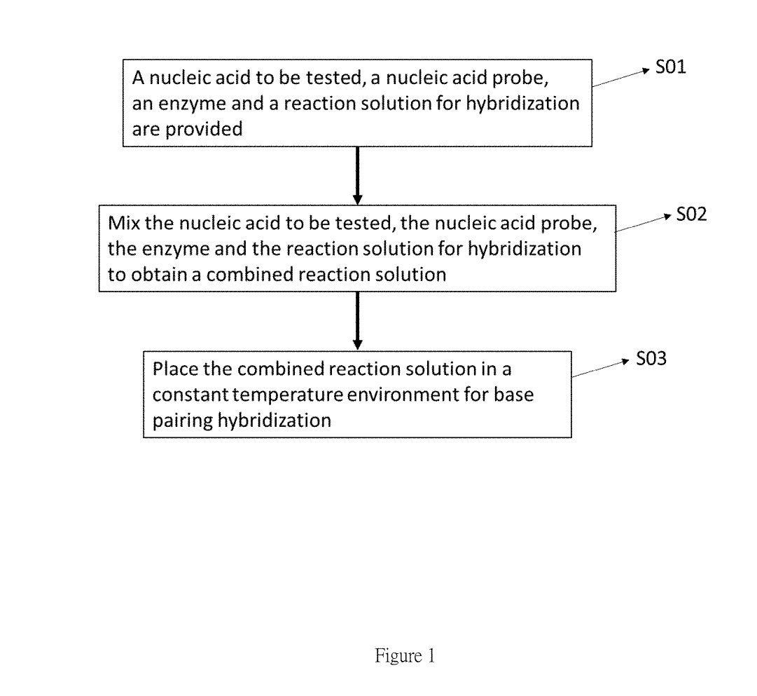

Refer to FIG. 1. Method of utilizing enzyme for isothermal nucleic acid hybridization in the invention comprises the following steps: step S01, a nucleic acid to be tested, a nucleic acid probe, an enzyme and a reaction solution for hybridization are provided, wherein the enzyme is recombinase; step S02, mix the nucleic acid to be tested, the nucleic acid probe, the recombinase and the reaction solution for hybridization to obtain a combined reaction solution; and step S03, place the combined reaction solution in a constant low temperature environment for base pairing hybridization, the low temperature is set between 30 to 45° C. Methods for proceeding base pairing nucleic acid hybridization comprise in situ hybridization, Southern blotting, Northern blotting and microarray hybridization. The nucleic acid to be tested can be single strand or double strand DNA or RNA. The nucleic acid probe may utilize but not limit to DNA or oligonucleotide probe and is labeled with radioactive isotope, such as 32p and 35s, or nonradioactive probe. Nonradioactive probe label comprises usage of metal, mercury (Hg) for example, fluorescein isothiocyanate (FITC), haptens, digitalin for example, biotin, enzymes, horseradish peroxidase (hrp) for example, galactosidase or alkaline phosphatase (akp). Preparation of Recombinase

Search target enzyme and DNA sequence correspond to the target enzyme in the database of National Center for Biotechnology Information; design a primer specifically for the DNA sequence then amplify the sequence by polymerase chain reaction (PCR); conform the length of PCR product by electrophoresis analysis then harvest the PCR product at the correct position from the agarose gel; and purify the

PCR product by gel extraction then proceed DNA sequencing of the purified PCR product to verify the correctness. The result of sequencing demonstrates as SEQ ID NO:44 in the Sequence Listing.

The purified PCR product and a suitable DNA vector were prepared to be a first plasmid by TA-cloning, the first plasmid has features of ampicillin resistance and mass duplication. The first plasmid was then transformed to E. coli DH5 a component cell. After transformation, E. coli was capable of mass duplicating the first plasmid, and therefore a great quantity of target DNA sequences are acquired. The first plasmid with target DNA sequences was extracted by plasmid extraction, and then restriction enzymes, 5′-NdeI and 3′-BamHI, were used to harvest the target DNA sequence from the first plasmid; meanwhile, a second vector (pET14b) was processed with the same restriction enzymes. The target DNA sequence and the second vector have the same cloning site; consequently, a second plasmid was formed by using ligase. The second plasmid was then transformed to BL21(DE3)pLysS component cell. T7 polymerase in the component cell with the second plasmid was activated by IPTG and mass target gene were transcribed, further, mass protein of the target gene were produced.

The transformed BL21(DE3)pLysS component cell was undergone mass culture and IPTG stimulation so that mass target protein could be acquired. Afterwards, lysis buffer was used to lyase the cells but maintain protein activity, and protein was further extracted and purified. The second plasmid in the invention was labelled 6× His tag and SUMO proteins (small ubiquitin-like modifier proteins). Specific antibody-coated beads were used to recognised 6× His tag for purification. Specific elution buffer was used to make antigen-antibody bond cleavage. The specific antibody-coated beads were removed by centrifugation or magnetic force. The amino acid sequence of target protein demonstrates as SEQ ID NO:45 in the Sequence Listing.

The target protein stated above is exactly the recombinase which was utilize in the DNA-DNA isothermal hybridization in the following two embodiments.

### Embodiment 1

Tuberculosis DNA (100 ng), E. coli DNA (100 ng) and eukaryote (human) A549 cell line DNA (100 ng) are nucleic acids to be tested. Tuberculosis specific probe (100 nmole), E. coli specific probe (100 nmole) and glyceraldehyde 3-phosphate dehydrogenase (GAPDH) specific probe (100 nmole) are nucleic acid probes with biotin labelled.

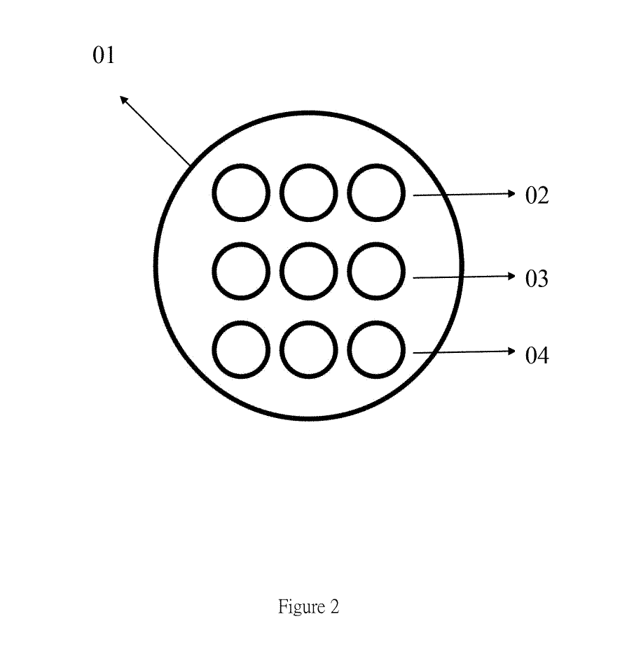

Refer to FIG. 2 which is a schematic drawing of the distribution of nucleic acid to be tested. The nucleic acids to be tested were fixed in a 96-well microtiter plate 01. The first row of the 96-well microtiter plate 01 was blotted with Tuberculosis nucleic acid 02, the second row of the 96-well microtiter plate 01 was blotted with E. coli nucleic acid 03, and the third row of the 96-well microtiter plate 01 was blotted with eukaryote A549 cell line nucleic acid 04. The nucleic acid probes (100 nmole), the recombinase (120 ng/μl) and the reaction solution for hybridization (50 μl, Tris (Ph 7.9) 50 mM, potassium acetate 100 mM, DTT 2 mM, PEG20K 5%, ATP 3 mM, phosphor creatine 50 mM, creatine kinase 100 ng/μl, MaAC 140 mM) were added into every microtiter well and mixed in the 96-well microtiter plate 01. Then the 96-well microtiter plate 01 was placed in a 40° C. environment to proceed hybridization for 30 minutes.

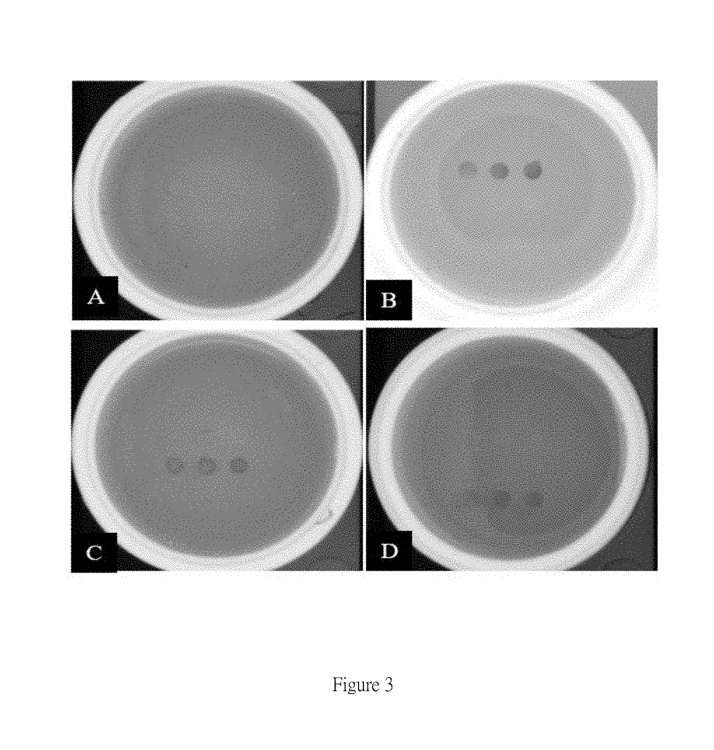

Unresponsive supernatant was removed, and redundant nucleic acid probes and reaction solution for hybridization were washed out three times with 500 μl rinsing solution (W)(0.1× SSC+0.1%SDS). Then 200 μl pigmentation solution (Latex with 5% bonded avidin) was added. Further, unresponsive supernatant was removed, and redundant pigmentation solution was washed out three times with 500 μl rinsing solution (W)(0.1× SSC+0.1%SDS). The results were shown in FIG. 3A to 3D.

Refer to FIG. 3A to 3D. FIG. 3A illustrates negative control group, in which any nucleic acid probe was not added. FIG. 3B illustrates the Tuberculosis specific probe (100 nmole) was added. FIG. 3C illustrates the E. coli specific probe (100 nmole) was added. FIG. 3D illustrates the GAPDH specific probe (100 nmole) was added. In view of FIG. 3B to 3D, the recombinase was able to combine the nucleic acid probes and unwind double stranded DNA so that the nucleic acid probes adhered to the nucleic acids to be tested via base pairing when correspondent nucleic acid sequences matched up. In addition, the nucleic acid probes were labelled with pigment and therefore the results could be interpreted by pigmentation reaction. This invention is capable of identifying to-be-tested DNA matching nucleic acid probe and then confirming if the to-be-tested DNA has target sequence. On the other hand, because there was no nucleic acid probe added in the negative control group in FIG. 3A, the recombinase did not serve non-specific base pairing function; for this reason, this invention is feasible and does not have false positive result.

### Embodiment 2

Embodiment 2 performed the detection of specific gene fragments of 36 types of human papilloma virus (HPV type 6, 11, 16, 18, 26, 31, 32, 33, 35, 39, 42, 43, 44, 45, 51, 52, 53, 54, 55, 56, 58, 59, 61, 62, 66, 67, 68, 69, 70, 72, 74, MM4, MM7, MM8, cp8061 and cp8304). HPV DNA in cervical smear was undergone nucleic acid amplification with MY11/GP6+ primer then undergone hybridization with HPV genotype specific oligonucleotide probe on a test sheet. Pigmentation reaction resulted from streptavidin conjugating with latex was used to interpret HPV genotype. Sequences of primers and nucleic acid probes used in the Embodiment 2 were demonstrated in the Sequence Listing. In the Sequence Listing, SEQ ID NO:1 is HPV-6 nucleic acid probe; SEQ ID NO:2 is HPV-11 nucleic acid probe; SEQ ID NO:3 is HPV-16(a) nucleic acid probe; SEQ ID NO:4 is HPV-18 nucleic acid probe; SEQ ID NO:5 is HPV-26 nucleic acid probe; SEQ ID NO:6 is HPV-31 nucleic acid probe; SEQ ID NO:7 is HPV-32 nucleic acid probe; SEQ ID NO:8 is HPV-33 nucleic acid probe; SEQ ID NO:9 is HPV-35 nucleic acid probe; SEQ ID NO:10 is HPV-39 nucleic acid probe; SEQ ID NO:11 is HPV-42 nucleic acid probe; SEQ ID NO:12 is HPV-43 nucleic acid probe; SEQ ID NO:13 is HPV-44 nucleic acid probe; SEQ ID NO:14 is HPV-45 nucleic acid probe; SEQ ID NO:15 is HPV-51 nucleic acid probe; SEQ ID NO:16 is HPV-52 nucleic acid probe; SEQ ID NO:17 is HPV-53 nucleic acid probe; SEQ ID NO:18 is HPV-54 nucleic acid probe; SEQ ID NO:19 is HPV-55 nucleic acid probe; SEQ ID NO:20 is HPV-56 nucleic acid probe; SEQ ID NO:21 is HPV-58 nucleic acid probe; SEQ ID NO:22 is HPV-59 nucleic acid probe; SEQ ID NO:23 is HPV-61 nucleic acid probe; SEQ ID NO:24 is HPV-62 nucleic acid probe; SEQ ID NO:25 is HPV-66 nucleic acid probe; SEQ ID NO:26 is HPV-67 nucleic acid probe; SEQ ID NO:27 is HPV-68 nucleic acid probe; SEQ ID NO:28 is HPV-69 nucleic acid probe; SEQ ID NO:29 is HPV-70 nucleic acid probe; SEQ ID NO:30 is HPV-72 nucleic acid probe; SEQ ID NO:31 is HPV-74 nucleic acid probe; SEQ ID NO:32 is HPV-MM4(82) nucleic acid probe; SEQ ID NO:33 is HPV-MM7(83) nucleic acid probe; SEQ ID NO:34 is HPV-MM8(84) nucleic acid probe; SEQ ID NO:35 is HPV-cp8061(71) nucleic acid probe; SEQ ID NO:36 is HPV-cp8304(81) nucleic acid probe; SEQ ID NO:37 is HPV-Pan I nucleic acid probe; SEQ ID NO:38 is HPV-Pan II nucleic acid probe; SEQ ID NO:39 is IC: GAPDH nucleic acid probe2; SEQ ID NO:40 is MY11 F-primer; SEQ ID NO:41 is biotin-HPV-R1 primer; SEQ ID NO:42 is GAPDH-F2 primer; SEQ ID NO:43 is biotin-GAPDH-R2 primer.

Specific nucleic acid probes of 36 HPV genotypes and DNA loading dye were mixed in the ratio of 10:1 and blotted on NC papers (sartorius stedim biotech, UniSart CN140 unbacked, cat NO: 1UN14AR10027ONT). The distribution matrix of the specific nucleic acid probes is as follows:

| | 1 | 2 | 3 | 4 | 5 | 6 | 7 | 8 | 9 | 10 | |
|---|---|---|---|---|---|---|---|---|---|---|---|
| A | Marker | | 32 | 53 | 68 | cp8304 | 67 | 52 | | Marker | A |
| B | | | 33 | 54 | 69 | cp8061 | 66 | 51 | | | B |
| C | | 6 | 35 | 55 | 70 | MM8 | 62 | 45 | 31 | baseline | C |
| D | Pan I | 11 | 39 | 56 | 72 | MM7 | 61 | 44 | 26 | | D |
| E | Pan II | 16 | 42 | 58 | 74 | MM4 (82) | 59 | 43 | 18 | | E |
| F | | 18 | 43 | 59 | MM4 (82) | 74 | 58 | 42 | 16 | IC | F |
| G | | 26 | 44 | 61 | MM7 | 72 | 56 | 39 | 11 | | G |
| H | baseline | 31 | 45 | 62 | MM8 | 70 | 55 | 35 | 6 | | H |
| I | | | 51 | 66 | cp8061 | 69 | 54 | 33 | | | I |
| J | Marker | | 52 | 67 | cp8304 | 68 | 53 | 32 | | IC | J |
| | 1 | 2 | 3 | 4 | 5 | 6 | 7 | 8 | 9 | 10 | |

Every test sheet contained 250 μl reaction solution for hybridization, reagent B, 25 μl GAPDH-nucleic acid amplified product and 25 μl HPV-nucleic acid amplified product, wherein the reaction solution comprised recombinase 20 IU and reagent A (2M GuSCN in PBS), and the reagent B comprised 5% BSA/0.05% NaN3/PBS, pH7.4-7.6, mixed in the ratio of 1:1, prepared right before usage. In the Embodiment 2, the nucleic acids to be tested were HPV type 33, 52, 56 and 58.

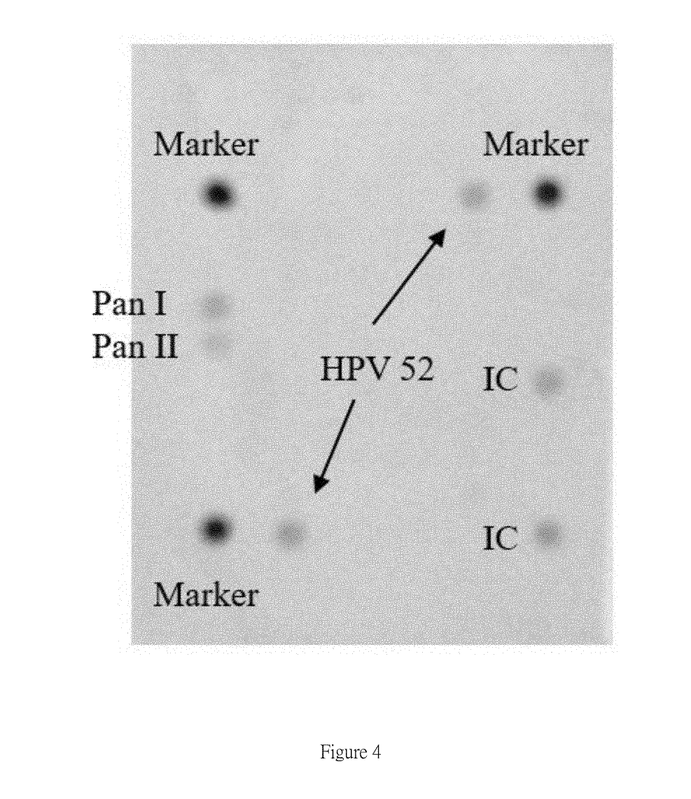

The reaction solution was hybridized for 30 minutes under 40° C. and 300 rpm oscillation and then washed out twice in 5 minutes with rinsing solution (1% Triton X-100 in PBS) under 37° C. and 300 rpm oscillation. Afterwards, 300 μl red latex pigment solution was added, and the hybridization continued for 5 minutes under 37° C. and 300 rpm oscillation. The red latex pigment solution was washed out twice in 5 minutes with 400 μl rinsing solution under 37° C. and 300 rpm oscillation. Test sheets were dried by heat for 10 minutes under 37° C. then identified HPV types by BP CHR-210 reader. The results were shown in FIGS. 4, 5 and 6.

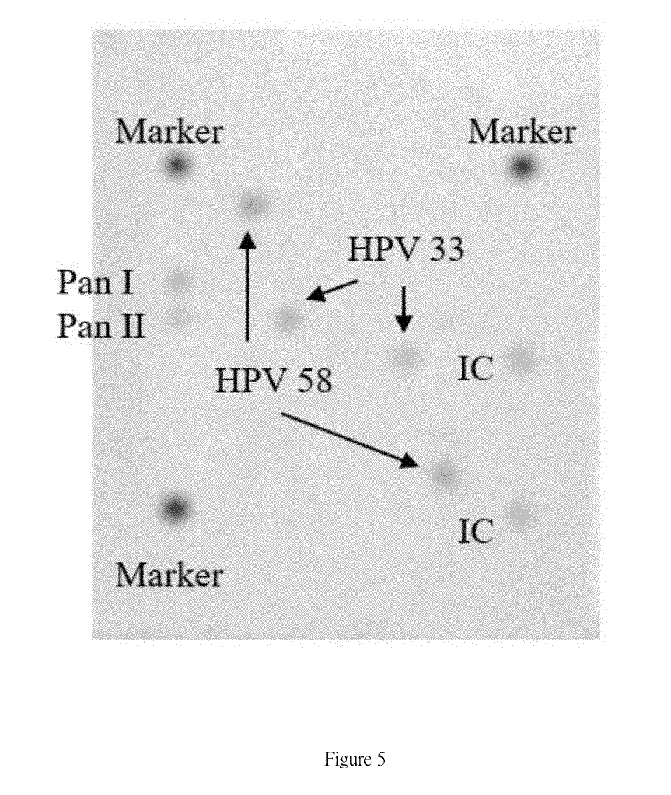

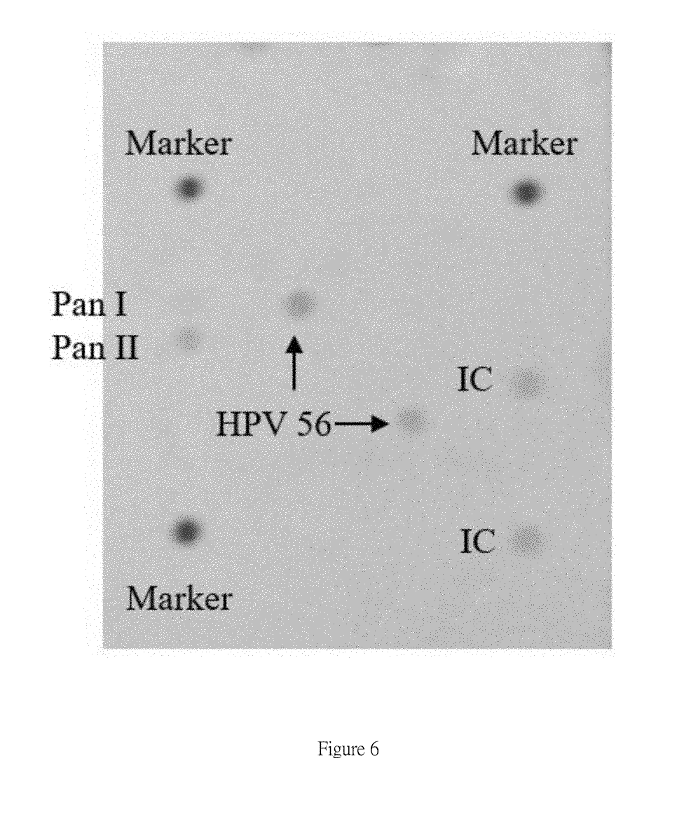

In FIG. 4, red dots arrows pointing indicate HPV type 52 nucleic acid. In FIG. 5, red dots arrows pointing indicate HPV type 33 and 58 nucleic acids. In FIG. 6, red dots arrows pointing indicate HPV type 56 nucleic acid.

In view of FIGS. 4, 5 and 6, the recombinase was able to combine the nucleic acid probes and unwind double stranded DNA so that the nucleic acid probes adhered to the nucleic acids to be tested via base pairing when correspondent nucleic acid sequences matched up. In addition, the nucleic acid probes were labelled with pigment and therefore the results could be interpreted by pigmentation reaction. This invention is capable of identifying to-be-tested DNA matching nucleic acid probe and then confirming if the to-be-tested DNA has target sequence. Besides, on account that there was no specific virus nucleic acids in other virus type sites, the recombinase did not serve non-specific base pairing function; for this reason, this invention is feasible and does not have false positive result.

The foregoing embodiments are illustrative of the characteristics of the present invention so as to enable a person skilled in the art to understand the disclosed subject matter and implement the present invention accordingly. The embodiments, however, are not intended to restrict the scope of the present invention. Hence, all equivalent modifications and variations made in the foregoing embodiments without departing from the spirit and principle of the present invention should fall within the scope of the appended claims.

What is claimed is:

1. A method of utilizing enzyme for isothermal nucleic acid hybridization, comprising steps of:
(1) providing a nucleic acid to be tested, a nucleic acid probe, an enzyme and a reaction solution for hybridization;
(2) mixing the nucleic acid to be tested, the nucleic acid probe, the enzyme and the reaction solution for hybridization to obtain a combined reaction solution; and
(3) placing the combined reaction solution in a constant temperature environment for base pairing hybridization.

2. The method as claimed in claim 1, wherein the enzyme is a recombinase which is capable of homologous nucleic acid base pairing and nucleic acid strand exchange.

3. The method as claimed in claim 1, wherein methods for proceeding the base pairing hybridization comprise in situ hybridization, Southern blotting, Northern blotting and microarray hybridization.

4. The method as claimed in claim 1, wherein the nucleic acid to be tested is single strand or double strand DNA or RNA.

5. The method as claimed in claim 1, wherein the nucleic acid probe is DNA or oligonucleotide probe.

6. The method as claimed in claim 5, wherein length of the nucleic acid probe is between 20 to 60 nucleotides.

7. The method as claimed in claim 5, wherein the nucleic acid probe is labeled with radioactive isotope or nonradioactive probe.

8. The method as claimed in claim 1, wherein temperature of the constant temperature environment is between 30° C. to 45° C.

9. The method as claimed in claim 1, wherein duration of the base pairing hybridization is between 10 to 60 minutes.
